SEQUENCE LISTING (17 sequences)

SEQ ID NO: 1 - protein, 184 aa, Homo sapiens
MHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 2 - protein, 192 aa, artificial sequence
  protein variant
MGGSHHHHHHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 3 - protein, 184 aa, artificial sequence
  protein variant
MHSHRDFQPVLHLVALNSPLSGGMRGIRGADFQCFQQARAVGLAGTFRAFLSSRLQDLYSIVRRADRAAVPIVNLKDELLFPSWEALFSASEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 4 - protein, 184 aa, artificial sequence
  protein variant
MHSHRDFQPVLHLVALNSPLSGGMRGIRGADFQCFQQARAVGLAGTFRAFLSSRLQDLYSIVRRADRAAVPIVNLKDELLFPSWEALFSASKAPLQPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 5 - protein, 183 aa, artificial sequence
  protein variant
MHSHRDFQPVLHLVALNSPLSGGMRGIRGADFQCFQQARAVGLAGTFRAFLSSRLQDLYSIVRRADRAAVPIVNLKDELLFPSWEALFSSEGPLKPGARIFSFDGRDVLRHPTWPQRSVWHGSDPNGRRLTESYCETWRTEAPSATGQASSLLGGRLLGQSAASCHHAYIVLCIENSFMTASR

SEQ ID NO: 6 - protein, 183 aa, artificial sequence
  protein variant
MHSHQDFQPVLHLVALNSPLSGGMRGIRGADFQCFQQARAVGLAGTFRAFLSSRLQDLYSIVRRADRAAVPIVNLKDELLFPSWEALFSSEGPLQPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 7 - protein, 181 aa, artificial sequence
  protein variant
MDFQPVLHLVALNSPLSGGMRGIRGADFQCFQQARAVGLAGTFRAFLSSRLQDLYSIVRRADRAAVPIVNLKDELLFPSWEALFSGESGAGKTPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 8 - protein, 180 aa, artificial sequence
  protein variant
MRDFQPVLHLVALNSPLSGGMRGIRGADFQCFQQARQVGLAGTFRAFLSSRLQDLYSIVRRADRAAVPIVNLKDELLFPSWEALFSSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 9 - protein, 181 aa, artificial sequence
  protein variant
MRDFQPVLHLVALNSPLSGGMRGIRGADFQCFQQARAVGLAGTFRAFLSSRLQDLYSIVRRADRGSVPIVNLKDEVLSPSWDSLFSGSQGQLQPGARIFSFDGRDILQDSAWPQKSVWHGSDAKGRRLPESYCEAWRTDERGTSGQASSLLSGRLLEQKAASCHNSYIVLCIENSFMTASK

SEQ ID NO: 10 - protein, 185 aa, artificial sequence
  protein variant
MHVHQDFQPALHLVALNTPLSGGMRGIRGADFQCFQQARQVGLAGTFRAFLSSRLQDLYSIVRRADRTAVPIVNLRDEVLFSNWEALFTGSEAPLRAGARIFSFDGRDVLRHPTWPQKSVWHGSDPNGRRLTESYCETWRTEAPSATGQASSLLAGRLLEQKAAGCHNAFIVLCIENSFMTSSSK

SEQ ID NO: 11 - protein, 186 aa, artificial sequence
  protein variant
MHTHQDFHPVLHLVALNTPLSGGMRGIRGADFQCFQQARAVGLSGTFRAFLSSRLQDLYSIVRRADRAAVPIVNLKDELLFPSWEALFSGESGAGKTGGARIFSFDGRDVLRHPAWPQKSVWHGSDPSGRRLTESYCETWRTDSRAATGQASSLLAGRLLEQKAAGCHNAFIVLCIENSFMTSSSK

SEQ ID NO: 12 - protein, 184 aa, artificial sequence
  protein variant
MHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGRDVLRHPTWPQKSVWHGSDPSGHRLTESYCETWRTDSRAATGQASSLLGGRLLGQSAASCHHAYIVLCIANSFMTASK

SEQ ID NO: 13 - protein, 180 aa, artificial sequence
  protein variant
MDFQPVLHLVALNSPLSGGMRGIRGADFQCFQQARAVGLAGTFRAFLSSRLQDLYSIVRRADRAAVPIVNLKDELLFPSWEALFSGSEGPLRPGARIFSFDGKDVLRHPTLPQKSVWHGSDPSGRRLTESYCETWRTDSRAATGQASSLLGGRLLGQSAASCHHAYIVLCIENSFMTASK

SEQ ID NO: 14 - protein, 181 aa, artificial sequence
  protein variant
MDFQPVLHLVALNSPLSGGMRGIRGADFQCFQQARAVGLAGTFRAFLSSRLQDLYSIVRRADRAAVPIVNLKDELLFPSWEALFSGSEGPLRPGARIFSFDGKDVLRHPTLPQKSVWHGSDPSGRRLTESYCETWRTDSRAATGQASSLLGGRLLGQSAASCHHAYIVLCIENSFMTASKK

SEQ ID NO: 15 - protein, 185 aa, artificial sequence
  protein variant
MHTHQDFHPVLHLVALNTPLSGGMRGIRGADFQCFQQARAVGLAGTFRAFLSSRLQDLYSIVRRADRAAVPIVNLKDELLFPSWEALFSGSEGPLKPGARIFSFDGRDILQDSAWPQKSVWHGSDPNGRRLTESYCETWRTEAPSATGQASSLSSGKLLEQSVSSCQHAFVVLCIENSFMTAAKK

SEQ ID NO: 16 - protein, 183 aa, artificial sequence
  protein variant
MHTHTSGPGLHLIALNSPQVGNMRGIRGADFQCFQQARAVGLAGTFRAFLSSRLQDLYSIVRRADRSSVPIVNLKDEVLSPSWDSLFSVSQGQLQPGARIFSFDGRDILQDSAWPQKSVWHGSDPNGRRLTESYCETWRTEAPSATGQASSLLGGRLLGQSAASCHHAYIVLCIENSFMTASK

SEQ ID NO: 17 - protein, 184 aa, artificial sequence
  protein variant
MTPTWYPRMLRVAALNEPSTGDLQGIRGADFQCFQQARAVGLSGTFRAFLSSRLQDLYSIVRRADRAAVPIVNLKDEVLSPSWDSLFSGSQGQLQPGARIFSFDGKDVLRHPTWPQKSVWHGSDPSGRRLMESYCETWRTETTGATGQASSLLGGRLLGQSAASCHHAYIVLCIENSFMTNNRK

(19) United States
(12) Patent Application Publication
(10) Pub. No.: US 2018/0015148 A1
(43) Pub. Date: Jan. 18, 2018

(54) DRUG FOR INHIBITING ADIPOSE CELL DIFFERENTIATION AND INSULIN RESISTANCE

(71) Applicants: TSINGHUA UNIVERSITY, Beijing (CN); BEIJING PROTGEN LTD., Beijing (CN)

(72) Inventors: Yongzhang LUO, Beijing (CN); Hui WANG, Beijing (CN); Hui LI, Beijing (CN); Xinan LU, Beijing (CN); Yan FU, Beijing (CN); Shunli ZHAN, Beijing (CN); Daifu ZHOU, Beijing (CN)

(73) Assignees: TSINGHUA UNIVERSITY, Beijing (CN); BEIJING PROTGEN LTD., Beijing (CN)

(21) Appl. No.: 15/524,094

(22) PCT Filed: Nov. 3, 2015
(86) PCT No.: PCT/CN2015/093726

§ 371 Date: Aug. 11, 2017

(30) Foreign Application Priority Data
Nov. 3, 2014 (CN) 201410610777.X
Jan. 15, 2015 (CN) 201510021469.8

(51) Int. Cl. A61K 38/39

(52) U.S. Cl. CPC A61K 38/39

(57) ABSTRACT

The present invention provides use of endostatin or a functional variant thereof in the preparation of a medicament for treating dietary obesity, non-alcoholic fatty liver disease, insulin resistance or glucose intolerance. In the embodiments of the present invention, the functional variant may be YH-16, mES, mYH-16, m003, m007, mZ101, or the like.

### FIELD OF THE INVENTION

The present invention relates to a novel function of endostatin. Specifically, the present invention discloses that endostatin significantly inhibits adipocyte differentiation and alleviates insulin resistance. The present invention also provides a new use of endostatin in treating dietary obesity, non-alcoholic fatty liver disease, insulin resistance, glucose intolerance, and other diseases.

### BACKGROUND OF THE INVENTION

The accumulation of fat could cause expansion of adipose tissue, which means increased number and volume of adipocytes along with angiogenesis (Cristancho A G et al., Nat Rev Mol Cell Biol 2011: 12:722-734; Daquinag A C et al., Trends Pharmacol Sci 2011; 32:300-307).

In 1997, Folkman's Laboratory discovered an endogenous vascular inhibitor endostatin (ES), which could be directly targeted to vascular endothelial cells, with angiogenesis inhibition and tumor treatment activities (O'Reilly M S et al., Cell 1997; 88:277-285: Boehm T. et al., Nature 197; 390:404-407).

YH-16 is an ES variant obtained by adding nine additional amino acids (MGGSHHHHH) at N-terminal of ES, which acquired national first-in-class new drug certificate in 2005 for the treatment of non-small cell lung cancer (Fu Y et al., IUBMB Life 2009; 61:613-626; Wang J et al., Zhongguo fei ai za zhi 2005; 8:283-290; Han B et al., J Thorac Oncol 2011: 6(6):1104-1109). PEG-modified ES and YH-16 were named as mES and mYH-16 respectively and were obtained by the modification of ES or YH-16 molecule with a 20 kDa monomethoxy polyethylene glycol propionaldehyde (mPEG-ALD). The coupling sites were activated aldehyde group of mPEG-ALD and N-terminal α-amino group of ES or YH-16.

It was reported that angiogenic inhibitor can inhibit obesity through inhibiting angiogenesis in adipose tissue (Rupnick M A et al., Proc Natl Acad Sci USA 2002: 99:10730-10735: Kim M Y et al., Int J Obes (Lond). 2010; 34:820-830). In 2002, Folkman's Laboratory reported several different vascular inhibitors that can inhibit hereditary obesity in mice, including ES (Rupnick M A et al., Proc Natl Acad Sci USA 2002: 99:10730-10735).

The increase in number of adipocytes directly depends on adipocyte differentiation (Cristancho A G et al., Nat Rev Mol Cell Biol 2011; 12:722-734), which is a very complicated regulatory process. Studies have shown that peroxisome proliferator-activated receptor gamma (PPARγ) is the central regulatory factor in regulating adipocyte differentiation (Tang Q Q et al., Annu Rev Biochem 2012; 81:715-736), which can regulate adipocyte differentiation by regulating the expression of downstream adipocyte phenotype control genes (including CD36, ap2, Glut4, LPL and LXR, etc.) (Cristancho A G et al., Nat Rev Mol Cell Biol 2011; 12:722-734: Lee J et al., J Cell Biochem 2012: 113:2488-2499).

A lot of epidemiological studies have shown that obesity can cause metabolic disorders, and is an important clinical manifestation of metabolic syndrome, but also an important risk factor in causing non-alcoholic fatty liver disease, insulin resistance, glucose intolerance, and type II diabetes (Malik V S et al., Nat Rev Endocrinol 2013: 9:13-27).

### SUMMARY OF THE INVENTION

The present invention relates to a novel function of the known vascular inhibitor protein endostatin (ES), namely the activity in inhibiting adipocyte differentiation, and provides, based on this novel function, a novel use of ES in the treatment of metabolic disorders such as dietary obesity, non-alcoholic fatty liver disease, insulin resistance, and glucose intolerance, etc.

The inventor discovered that ES can inhibit adipocyte differentiation by acting directly on preadipocytes and inhibiting the expression of central regulatory factors PPARγ1 and/or PPARγ2 in adipocyte differentiation.

The inventor discovered that ES can inhibit weight gain induced by high-fat diet in mice by inhibiting the accumulation of fat in mice.

The inventor discovered that ES can inhibit the increase in liver weight and fat deposition induced by high-fat diet in mice, thereby preventing and treating hepatic adipose infiltration.

The inventor also discovered that ES can enhance the response of mice to insulin by increasing the phosphorylation of Akt, so as to improve insulin resistance and glucose intolerance in mice.

The inventor also discovered that the variants of ES, such as YH-16, 003, 007 and Z101, have activity comparable to that of ES in above experiments. Polyethylene glycol (PEG)-modified ES and its variants YH-16, 003, 007 and Z101 (mES, mYH-16, m003, m007 and mZ101) have similar activity to the unmodified protein.

### BRIEF DESCRIPTION OF THE DRAWINGS

FIG. 1A shows that ES and its variant YH-16 significantly inhibited weight gain induced by high-fat diet in mice. *** means P<0.001.

FIG. 1B shows that ES and its variant YH-16 significantly inhibited the increase in adipose tissue weight induced by high-fat diet in mice. * means P<0.05, *** means P<0.001.

FIG. 1C shows that ES and its variant YH-16 had no effect on the weight of lungs, heart and kidneys in mice with high-fat diet.

FIG. 2A shows that ES and its variant YH-16 significantly inhibited the increase in liver weight induced by high-fat diet in mice. * means P<0.05, ** means P<0.01.

FIG. 2B shows that ES and its variant YH-16 treated group mice liver tissue slices.

FIG. 2C shows that ES and its variant YH-16 significantly inhibited liver fat deposition induced by high-fat diet in mice. *** means P<0.001.

FIG. 3A shows that ES and its variant YH-16 significantly improved insulin resistance in mice. *** means P<0.001.

FIG. 3B shows that ES and its variant YH-16 significantly improved glucose tolerance in mice. *** means P<0.001.

FIG. 3C shows that ES significantly increased the phosphorylation level of Akt, the downstream factor of insulin signaling pathway.

FIG. 4A shows that ES and its variant YH-16, PEG-modified ES and its variant YH-16 (mES and mYH-16) directly inhibited adipocyte differentiation.

FIG. 4B shows that quantitative statistical results of inhibition of adipocyte differentiation by ES and its variant YH-16, PEG-modified ES and its variant YH-16 (mES and mYH-16). *** means P<0.001

FIG. 4C shows that ES and its variant YH-16, PEG-modified ES and its variant YH-16 (mES and mYH-16) significantly inhibited protein expression of adipocyte differentiation central regulatory factors PPARγ1/2.

FIG. 4D shows that ES inhibited mRNA expression level of adipocyte differentiation central regulatory factors PPARγ1/2. * means P<0.05, *** means P<0.001.

FIG. 5A shows that PEG-modified ES and its variants 003, 007 (mES, m003, and m007) significantly inhibited weight gain induced by high-fat diet in mice. *** means P<0.001.

FIG. 5B shows that PEG-modified ES and its variants 003, 007 (mES, m003, and m007) significantly inhibited the increase in adipose tissue weight induced by high-fat diet in mice. * means P<0.05. *** means P<0.001.

FIG. 5C shows that PEG-modified ES and its variants 003, 007 (mES, m003, and m007) had no effect on the weight of lungs, heart and kidneys in mice with high-fat diet.

FIG. 6A shows that PEG-modified ES and its variants 003, 007 (mES, m003, and m007) significantly inhibited the increase liver weight induced by high-fat diet in mice. * means P<0.05, ** means P<0.01.

FIG. 6B shows that PEG-modified ES and its variants 003, 007 (mES, m003, and m007) treated group mice liver tissue slices.

FIG. 6C shows that PEG-modified ES and its variants 003, 007 (mES, m003, and m007) significantly inhibited liver fat deposition induced by high-fat diet in mice. *** means P<0.001.

FIG. 7A shows that PEG-modified ES and its variants 003, 007 (mES, m003, and m007) directly inhibited adipocyte differentiation.

FIG. 7B shows that quantitative results of inhibition of adipocyte differentiation by PEG-modified ES and its variants 003, 007 (mES, m003, and m007). *** means P<0.001.

FIG. 8A shows that PEG-modified ES variant Z101 (mZ101) significantly inhibited weight gain induced by high-fat diet in mice. ** means P<0.01, *** means P<0.001.

FIG. 8B shows that PEG-modified ES variant Z101 (mZ101) significantly inhibited the increase in adipose tissue weight induced by high-fat diet in mice. *** means P<0.001.

FIG. 8C shows that PEG-modified ES variant Z101 (mZ101) significantly inhibited the increase in liver weight induced by high-fat diet in mice. * means P<0.05.

FIG. 8D shows that PEG-modified ES variant Z101 (mZ101) had no effect on the weight of lungs, heart and kidneys in mice with high-fat diet.

FIG. 9A shows that PEG-modified ES variant 2101 (mZ101) directly inhibited adipocyte differentiation.

FIG. 9B shows that quantitative results of inhibition of adipocyte differentiation by PEG-modified ES variant Z101 (mZ101). *** means P<0.001.

FIG. 10A shows that PEG-modified ES variants 009 and S03 (m009 and mS03) significantly inhibited weight gain induced by high-fat diet in mice. ** means P<0.01, *** means P<0.001.

FIG. 10B shows that PEG-modified ES variants 009 and S03 (m009 and mS03) significantly inhibited the increase in adipose tissue weight induced by high-fat diet in mice. *** means P<0.001.

FIG. 10C shows that PEG-modified ES variants 009 and S03 (m009 and mS03) significantly inhibited the increase in liver weight induced by high-fat diet in mice. * means P<0.05.

FIG. 10D shows that PEG-modified ES variants 009 and S03 (m009 and mS03) had no effect on the weight of lungs, heart and kidneys in mice with high-fat diet.

FIG. 11A shows that PEG-modified ES variants 009 and S03 (m009 and mS03) directly inhibited adipocyte differentiation.

FIG. 11B shows that quantitative results of inhibition of adipocyte differentiation by PEG-modified ES variants 009 and S03 (m009 and mS03). *** means P<0.001.

FIG. 12A shows that PEG-modified ES variants 36 and 249 (m36 and m249) significantly inhibited weight gain induced by high-fat diet in mice. ** means P<0.01, *** means P<0.001.

FIG. 12B shows that PEG-modified ES variants 36 and 249 (m36 and m249) significantly inhibited the increase in adipose tissue weight induced by high-fat diet in mice. * means P<0.05, ** means P<0.01, *** means P<0.001.

FIG. 12C shows that PEG-modified ES variants 36 and 249 (m36 and m249) significantly inhibited the increase in liver weight induced by high-fat diet in mice. * means P<0.05, ** means P<0.01.

FIG. 12D shows that PEG-modified ES variants 36 and 249 (m36 and m249) had no effect on the weight of lungs, heart and kidneys in mice with high-fat diet.

FIG. 13 shows the amino acid sequence of ES variant 003.

FIG. 14 shows the amino acid sequence of ES variant 007.

FIG. 15 shows the amino acid sequence of ES variant Z101.

FIG. 16 shows the amino acid sequence of ES variant 009.

FIG. 17 shows the amino acid sequence of ES variant S03.

FIG. 18 shows the amino acid sequence of ES variant 36.

FIG. 19 shows the amino acid sequence of ES variant 249.

FIG. 20 shows the amino acid sequence of ES.

FIG. 21 shows the amino acid sequence of ES variant YH-16.

FIG. 22 shows the amino acid sequences of ES variants 381, 57, 114, and 124.

FIG. 23 shows the amino acid sequences of ES variants 125, 160, 163, and 119.

### DETAILED DESCRIPTION OF THE INVENTION

The present invention provides use of endostatin or a functional variant thereof in preparing a medicament for treating dietary obesity, non-alcoholic fatty liver disease, insulin resistance or glucose intolerance.

The present invention provides use of endostatin or a functional variant thereof in preparing a medicament for preventing adipocyte differentiation.

In some embodiments, the said functional variant may be YH-16, 003,007, Z101, ES006, ES008, ES011. S02, S09, Z006, Z008, ZN1, 009, S03, 36, 249, 381, 57, 114, 124, 125, 160, 163, 119, mES, mYH-16, m003, m007, mZ101, mES006, mES008, mES011, mS02, mS09, mZ006, mZ008, mZN1, m009, mS03, m36, m249, m381, m57, m114, m124, m125, m160, m163, or m119. In preferred embodiments of the present invention, the said functional variant may be YH-16, 003, 007, Z101, 009, S03, 36, 249, mES, mYH-16, m003, m007, mZ101, m009, mS03, m36, or m249.

As used herein, the terms “functional variant” and “functional variants” include endostatin mutants having substitution, deletion or addition of one or more (for example 1 to 5, 1 to 10 or 1 to 15, specifically, for example, 1, 2, 3, 4, 5, 6, 7, 8, 9, 10, 12 or even more) amino acids in the amino acid sequence, and derivatives obtained by chemically modifying endostatin or its mutants, for example, PEG modification. The mutants and derivatives have substantially the same activity of inhibiting adipocyte differentiation as endostatin. For example, PEG-modified ES and YH-16 are named as mES and mYH-16 respectively, and are obtained by the modification of ES or YH-16 with a 20 kDa monomethoxy polyethylene glycol propionaldehyde (mPEG-ALD). The coupling sites are activated aldehyde group of mPEG-ALD and N-terminal α-amino group of ES or YH-16 (other ES mutants and PEG-modified derivatives of the mutants are similarly modified and named). For example, in the embodiments of the present invention, YH-16, 003, 007, Z101, 009, S03, 36 and 249 are the particularly preferred mutants of endostatin; mES, mYH-16, m003, m007, mZ101, m009, mS03, m36 and m249 are preferred derivatives of ES, YH-16, 003, 007, Z101, 009, S03, 36 and 249 respectively. PCT application PCT/CN2012/081210 (which is hereby incorporated by reference in its entirety) provides various mutants of endostatin such as ES006, ES008. ES011, S02, S09, Z006, Z008, and ZN1 etc. The terms “functional variant”, “functional variants”, “variant”, or “variants” in this context cover the mutants and derivatives of endostatin.

The present invention also provides a method for treating dietary obesity, non-alcoholic fatty liver disease, insulin resistance or glucose intolerance, comprising administering to a subject a therapeutically effective amount of endostatin or a functional variant thereof.

As used herein, the term “therapeutically effective amount” refers to an amount of active compound sufficient to cause a biological or medical response desired by the clinician in a subject. The “therapeutically effective amount” of endostatin or a functional variant thereof can be determined by those skilled in the art depending on factors such as route of administration, weight, age and condition of the subject, and the like. For example, a typical daily dose may range from 0.01 mg to 100 mg of active ingredient per kg of body weight.

The medicament provided in the present invention can be prepared into a clinically acceptable dosage form such as a powder, an injection and the like, and can be administered by conventional means such as injection.

The present invention also provides a method for inhibiting adipocyte differentiation; comprising administering to a subject a therapeutically effective amount of endostatin or a functional variant thereof.

The present invention also provides a medicament for the treatment of dietary obesity, non-alcoholic fatty liver disease, insulin resistance or glucose intolerance, including endostatin or a functional variant thereof as active ingredient.

Dietary obesity refers to obesity caused by excess calories stored in the form of fat in the body when the calories in the diet exceed the body's energy consumption.

Non-alcoholic fatty liver disease (NAFLD) is metabolic stress induced liver injury closely correlated with insulin resistance and genetic susceptibility. Its pathological phenotype is similar to that of alcoholic liver disease (ALD), but patients have no history of excessive drinking.

Insulin resistance, also known as insulin tolerance, which means the insusceptibility of body to insulin so that the promoting effect of insulin on the intake and utilization of glucose is below normal level. In other words, the body requires higher concentration of insulin to respond to insulin. Insulin resistance induced high level of insulin and high glucose in the plasma usually lead to metabolic syndrome, gout and type II diabetes.

Glucose intolerance is the decline in the capability to adjust blood glucose level due to the reduced glucose metabolism of the body, manifested in that the blood glucose level cannot be timely adjusted back to normal after large intake of glucose. It can develop into diabetes if not interfered timely.

Inhibitory ratio of mice body weight=(1−increase of body weight in the drug treated group/increase of body weight in the group with high-fat diet)×100%.

Inhibitory ratio of mice fat storage=(1−adipose tissue weight in the drug treated group/adipose tissue weight in the group with high-fat diet)×100%.

Inhibitory ratio of mouse liver weight=(1−liver weight in the drug treated group/liver weight in the group with high-fat diet)×100%.

Inhibitory ratio of mouse liver fat deposition=(1−hepatic cytoplasmic vacuolar ratio in the drug treated group/hepatic cytoplasmic vacuolar ratio in the group with high-fat diet)×100%.

The ES and variants thereof utilized in the examples of the present invention were all provided by Beijing Protgen Ltd.

### EXAMPLES

### Example 1 ES and YH-16 Significantly Inhibited Weight Gain Induced by High-Fat Diet in Mice

A total of 24 healthy C57BL/6 mice (7-week old, male, purchased from Beijing Vital River Laboratory Animal Technology Company) were divided into 4 groups with 8 mice in each group and treated as follows:

Group 1: normal diet group;

Group 2: high-fat diet group;

Group 3: high-fat diet+ES treated group (drug treated group);

Group 4: high-fat diet+YH-16 treated group (drug treated group).

Mice in normal diet group were fed with feedstuff in which 10% calories come from fat component (D12450J, Research Diets, USA): mice in high-fat diet group were fed with feedstuff in which 60% calories come from fat component (D12492J, Research Diets, USA).

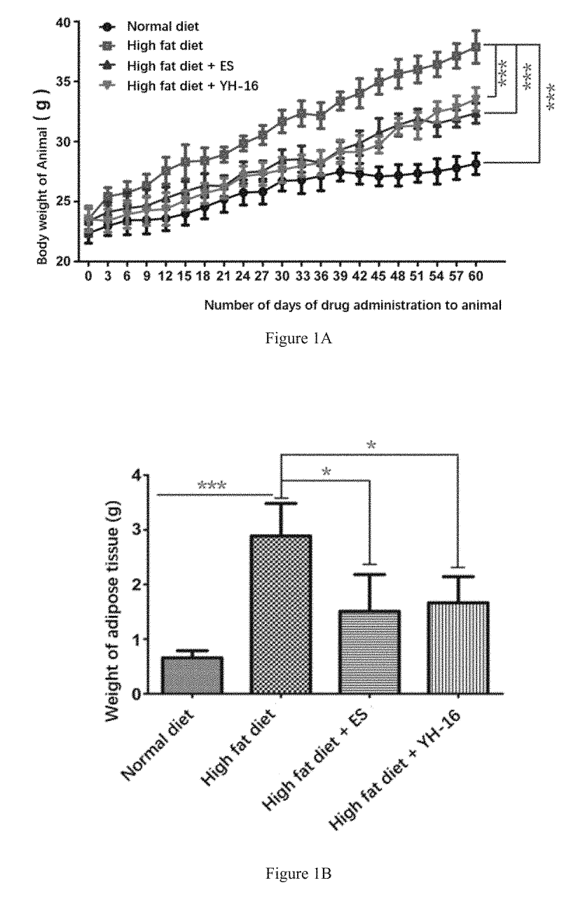

Route of administration: in an injection period of 60 days, group 3 and group 4 were injected intraperitoneally once a day with ES or YH-16 (Protgen) at a dose of 12 mg/kg/day, group 2 was injected intraperitoneally with equal volume of saline, group 1 was not injected. The first day of injection was set as day 0, and the last administration was carried out on day 59. The mice were weighed once every three days, and the last measurement was performed on day 60 (i.e., the day after the last administration), and the body weight curves were plotted (FIG. 1A). The results showed that both ES and YH-16 significantly inhibited weight gain due to high-fat diet, and the inhibition ratios were 37.5%, and 30.6% respectively (Table 1).

After completion of the glucose tolerance test on day 61, the mice were sacrificed and whole body adipose tissues were isolated and weighed (FIG. 1B, Table 1). The results showed that the adipose tissue weight of mice in group with ES or YH-16 treatment was remarkably lower than that in high-fat diet group without drug treatment. The inhibitory ratios of ES and YH-16 on fat accumulation induced by high-fat diet in mice were 47.7%, and 42.2%/0, respectively (Table 1).

Lungs, heart and kidneys were isolated from mice and weighed (FIG. 1C, Table 1). The results showed that there was no significant difference in the weight of lungs, heart and kidneys among the mice in all four groups, indicating that ES and YH-16 had no effect on lungs, heart and kidneys of mice.

### Example 2 ES and YH-16 Significantly Inhibited the Increase in Liver Weight and Fat Deposition Induced by High-Fat Diet in Mice

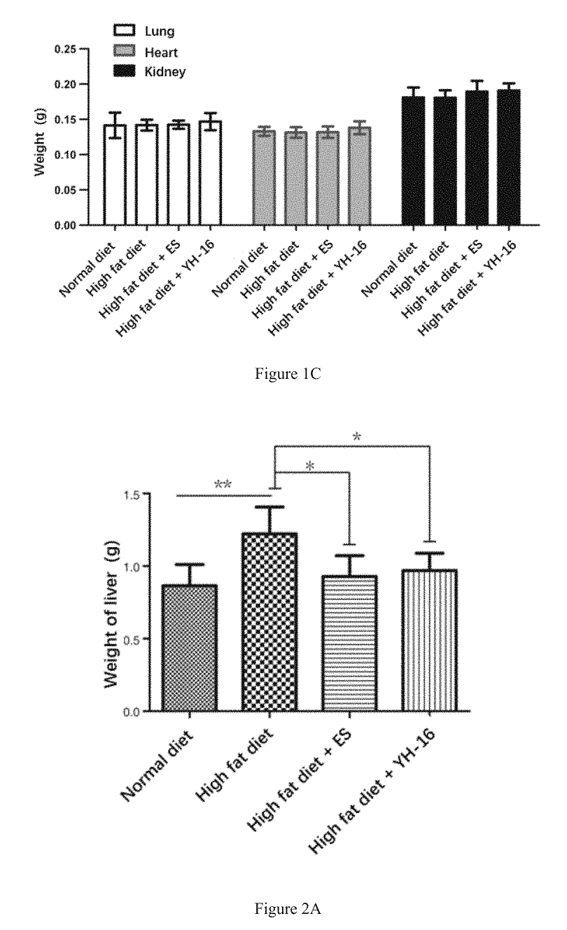

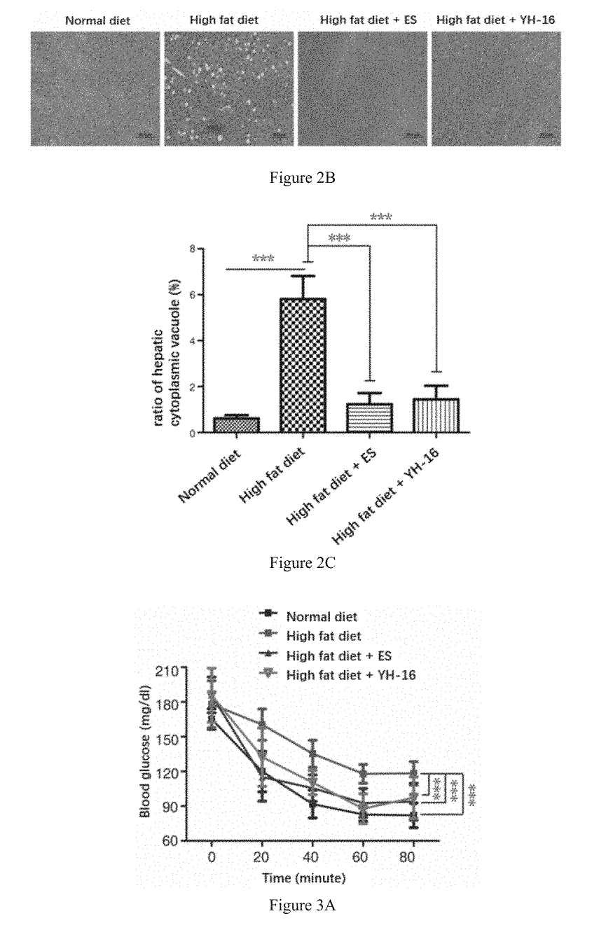

From the mice in Example 1, after completion of the glucose tolerance test on day 61, the liver tissues were removed and weighed (FIG. 2A, Table 1). ES and YH-16 inhibited the increase in liver weight induced by high-fat diet in mice, with inhibition ratios of 23.8% and 20.5%, respectively.

The liver tissues were fixed and embedded in paraffin, then sliced into 8 μm thick sections. Then the liver tissue samples were stained with hematoxylin and eosin (HE). Major steps included: after deparaffination and rehydration, the sections were stained with hematoxylin and eosin, followed by conventional dehydration, and sealing, then observed with conventional optical microscope (Olympus IX71 microscope) and photographed (FIG. 2B). HE staining results showed that there were hepatic cytoplasmic vacuoles in liver tissue sections from mice in high-fat diet group, indicating that high-fat diet could cause fat deposition in liver, while the fat deposition in livers from mice in ES and YH-16 treated groups were significantly lower than that in high-fat diet group without drug administration, with inhibition ratios of 78.9%, and 75.2%, respectively (FIG. 2C). This indicated that ES and YH-16 have a significant inhibitory effect on non-alcoholic fat liver disease.

### Example 3 ES and YH-16 Significantly Improved Insulin Resistance and Glucose Intolerance in Mice

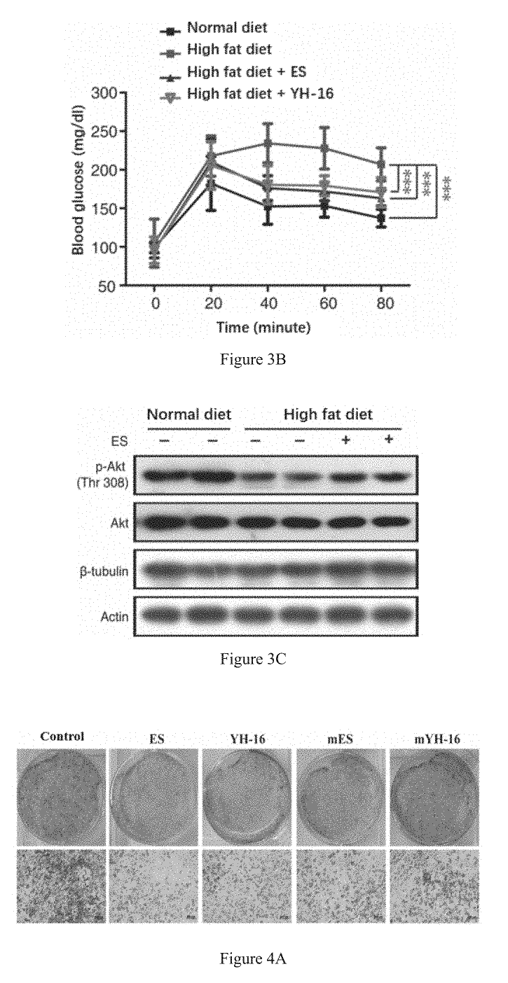

The mice in Example 1 were subjected to an insulin tolerance test 6 hours after completion of administration on day 59. Specific steps included: the tails of mice were cut and blood was collected, basic blood glucose concentrations were measured (Roche hand-held blood glucose meter), and the monitoring time was set to 0 minute. Biosynthetic human insulin (Novolin R, Novo Nordisk) was injected intraperitoneally at 0.5 U/kg, blood samples were taken at 20 min, 40 min, 60 min, 80 min after injection of insulin, and the blood glucose concentrations were measured and a curve was plotted. (FIG. 3A). It was found that after insulin injection, the blood glucose levels of mice in normal diet group quickly reduced over time, while the blood glucose levels of mice in high-fat diet group reduced slowly, indicating that high-fat diet could cause insulin resistance, and ES and YH-16 could significantly alleviate insulin resistance caused by high-fat diet.

The mice in Example 1, after weighing on day 60, were subjected to starvation overnight and the glucose tolerance test was performed on day 61. Specific steps included: the tails of mice were cut and blood was collected, basic blood glucose concentrations were measured (Roche hand-held blood glucose meter), and the monitoring time was set to 0 minute. The mice were fed by gavage with glucose solution (20 mg/ml), at a dose of 1 mg of glucose per gram of body weight of each mouse. Blood samples were taken at 20 min, 40 min, 60 min, 80 min after the gavage with glucose, and the blood glucose levels of mice were measured and a curve was plotted. (FIG. 3B). It was found that after the gavage with glucose, with the passage of time, the blood glucose level of the mice increased rapidly and the recovery rate was slow in high-fat diet group compared to the normal diet group, indicating that high-fat diet could lead to glucose intolerance in mice, while glucose intolerance in mice of ES and YH-16 treated group was significantly improved.

After completion of the glucose tolerance test on day 61, the mice were sacrificed and whole body adipose tissues were isolated, then the phosphorvylation levels of Akt in adipose tissues were detected by Western blot (FIG. 3C). The results showed that compared with normal diet group, the phosphorylation level of Akt was lower in high-fat diet group, while the phosphorylation level of Akt in ES treated group was higher than that in high-fat diet group. Akt pathway is an important blood glucose regulatory pathway downstream of insulin. Insulin resistance often accompanies with decreased Akt phosphorylation level. This is consistent with the fact that ES could effectively improve insulin resistance and glucose intolerance.

### Example 4 ES and YH-16 Significantly Inhibited the Differentiation of Preadipocytes into Adipocytes

3T3-L1 preadipocytes in good condition were selected and resuspended in DMEM medium supplemented with 10% FBS, then seeded into six-wells plate, and conventionally incubated at 37° C., 5% CO₂ in an incubator. The cells grew for two days, then began to induce differentiation: Step 1. MDI induction medium was added for induction (defining the time as day 1 of cell differentiation): Step 2. two days later, the medium was changed to insulin induction medium, and continued to culture for two more days. Step 3. the medium was changed to DMEM medium supplemented with 10% FBS, and continued to culture until day 8, 3T3-L1 were differentiated into adipocytes. This experiment was divided into 5 groups:

Group 1: control group:

Group 2: ES treated group;

Group 3: YH-16 treated group;

Group 4: mES treated group;

Group 5: mYH-16 treated group.

Among them, drug treated groups were supplemented with 50 μg/ml ES, YH-16, mES or mYH-16 during induction (i.e. day 1 to day 8), control group was added with equal volume of protein buffer. Aforesaid drug supplement and control treatment were carried out at each time when the medium was replaced.

MDI induction medium was prepared by adding 1 μM Dexamethasone, 0.5 mM 3-isobutyl-1-methylxanthine and 10 μg/ml bovine insulin into DMEM medium supplemented with 10% FBS. Insulin induction medium was prepared by adding 10 μg/ml bovine insulin into DMEM medium supplemented with 10% FBS.

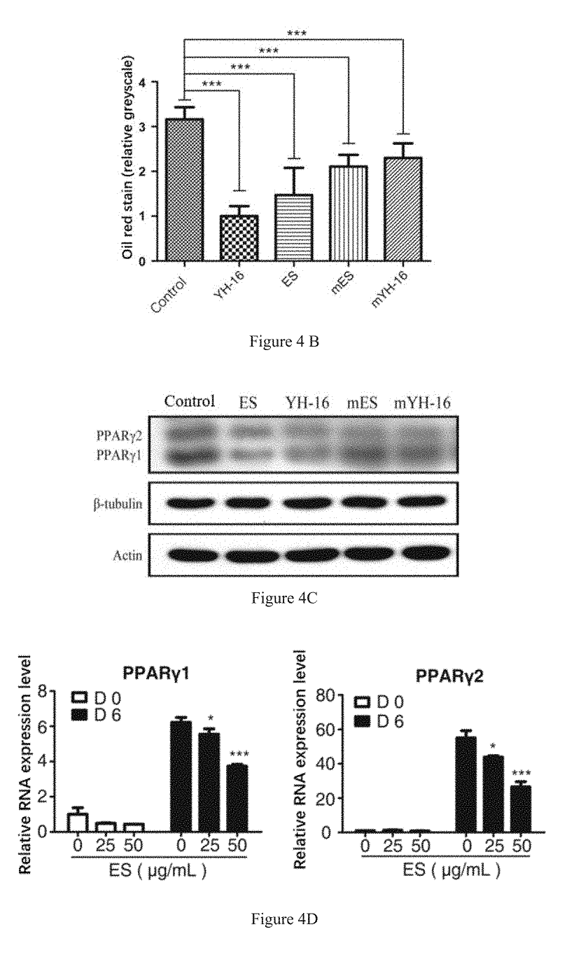

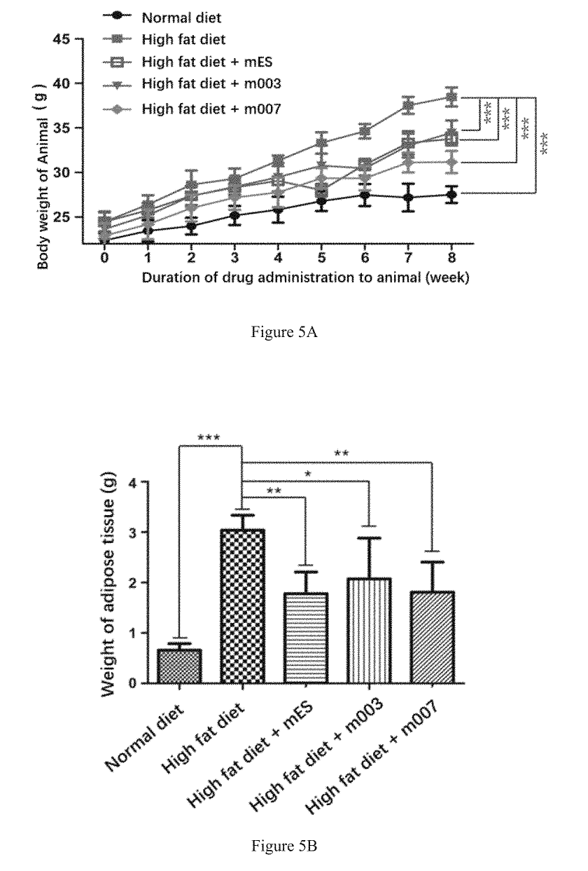

After induction, the medium in the six-well plate was removed, and the cells were fixed and stained with Oil red for 10 minutes. Then the cells were decolorized and rinsed three times with PBS to remove excess dye. Fats in adipocyte could be identified by Oil red, and then stained to red. The six-well plate was photographed with a digital camera, also observed and recorded by photograph with an invert microscope (Olympus IX71 microscope) (FIG. 4A). The results showed that ES, YH-16, mES and mYH-16 all could inhibit the differentiation of preadipocytes into adipoctes (FIG. 4B).

The cells were harvested on day 6 of the induction process, with medium removed. 100 μl 2×SDS electrophoretic loading buffer was added, and the cells were heated at 100° C. for 15 minutes. After electrophoresis and film transfer, the expression levels of PPARγ1 and PPARγ2, the central control factors in adipocyte differentiation, in whole cell lysates from each group were detected by immunoblotting (FIG. 4C). It was found that in adipocyte differentiation, ES, YH-16, mES and mYH-16 all could inhibit the protein expression levels of PPARγ1 and PPARγ2, both of which are the central control transcription factors in adipocyte differentiation.

Detection of PPARγ1 and PPARγ2 mRNA expression levels: the total RNA of 3T3-L1 was extracted according to the standard protocol of TRIZOL reagent (purchased from Invitrogen) protocol prior to induction (day 0) and on day 6 of induction. Fermentas reverse transcription kit (RevertAid™ First Stand cDNA Synthesis Kits) was used for reverse transcription according to the standard protocol.

PPARγ1/2, central control factor of adipocyte differentiation, was detected by fluorescence quantitative Real-Time PCR with Stratagene kit (Brilliant 11 SYBR®Green QRT-PCR Master Mix). MX3000P (purchased from Stratagene) as fluorescence quantitative PCR instrument, SYBR Green as fluorescent dye, with PCR reaction system of 20 μl, and reaction cycles of 40.

PCR procedure: denaturing at 95° C., 10 s; annealing and extending at 60° C., 30 s: reaction system of 20 μl, reaction cycles of 40; finally keeping at 75° C., 5 minutes. GAPDH was used as internal reference. The reaction primers were as follows:

PPARγ1 forward primer (5′-3′):
ACAAGATTTGAAAGAAGCGGTGA
PPARγ1 reverse primer (5′-3′):
GCTTGATGTCAAAGGAATGCGAAGGA
PPARγ2 forward primer (5′-3′):
CGCTGATGCACTGCCTATGAG
PPARγ2 reverse primer (5′-3′):
TGGGTCAGCTCTTGTGAATGGAA
GAPDH forward primer (5′-3′):
CCAGCCTCGTCCCGTAGACA
GAPDH reverse primer (5′-3′):
TGAATTTGCCGTGAGTGGAGTC

Using GAPDH as internal reference, ΔCt values were obtained according to the fluorescence diagram given by the instrument, then relative Δ(ΔCt) values were calculated, and then relative changes in mRNA levels of PPARγ1 and PPARγ2 were calculated (FIG. 4D). It was found that ES could inhibit the mRNA expression level of PPARγ1 and PPARγ2, the central control transcription factors, in adipocyte differentiation.

### Example 5 PEG-Modified ES and its Variants 003 and 007 (mES, m003, and m007) Significantly Inhibited Weight Gain Induced by High-Fat Diet in Mice

A total of 40 healthy C57BL/6 mice (7-week old, male, purchased from Beijing Vital River Laboratory Animal Technology Company) were divided into 5 groups with 8 mice in each group, and treated as follows:

Group 1: normal diet group:

Group 2: high-fat diet group:

Group 3: high-fat diet+mES treated group (drug treated group),

Group 4: high-fat diet+m003 treated group (drug treated group),

Group 5: high-fat diet+m007 treated group (drug treated group).

The diets for each group were the same as in Example 1.

Route of administration: in a period of 8 weeks, group 3, group 4 and group 5 were injected with mES, m003 or m007 (Protgen) via tail vein once a week, at a dose of 50 mg/kg/week, group 2 was injected with equal volume of saline, and group 1 was not injected. The first time of injection was set as week 0, the last administration was carried out on week 7. The mice were weighed once a week, and after the last measurement in week 8 the body weight curves were plotted (FIG. 5A). The results showed that mES, m003, and m007 significantly inhibited weight gain due to high-fat diet, and the inhibition ratios were 33.7%, 22.9%, and 42.9%, respectively (Table 2).

After the last mice body weight measurement in week 8, the mice were sacrificed and whole body adipose tissues were isolated and weighed (FIG. 5B. Table 2). The results showed that the adipose tissue weight of mice in mES, m003, and m007 groups were significantly lower than that in high-fat diet group without drug treatment. The inhibition ratios of mES, m003, and m007 on fat accumulation induced by high-fat diet in mice were 41.4%, 31.9%, and 40.5%, respectively (Table 2).

Lungs, heart and kidneys were isolated from mice and weighed (FIG. 5C, Table 2). The results showed that there was no significant difference in the weight of lungs, heart and kidneys among the mice in all five groups, indicating that mES, m003, and m007 had no effect on lungs, heart and kidneys of mice.

### Example 6 PEG-Modified ES and its Variants 003 and 007 (mES, m003, and m007) Significantly Inhibited the Increase in Liver Weight and Fat Deposition Induced by High-Fat Diet in Mice

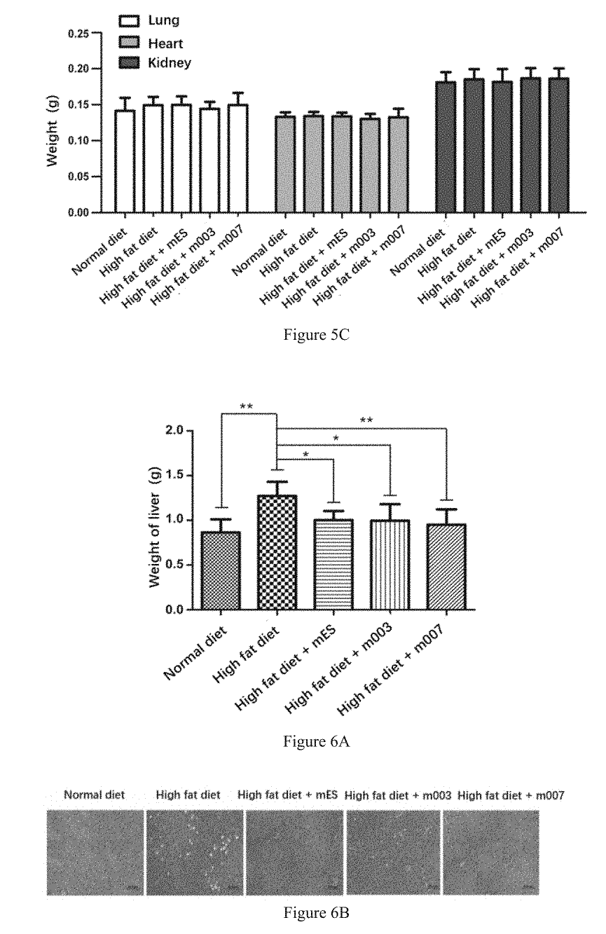

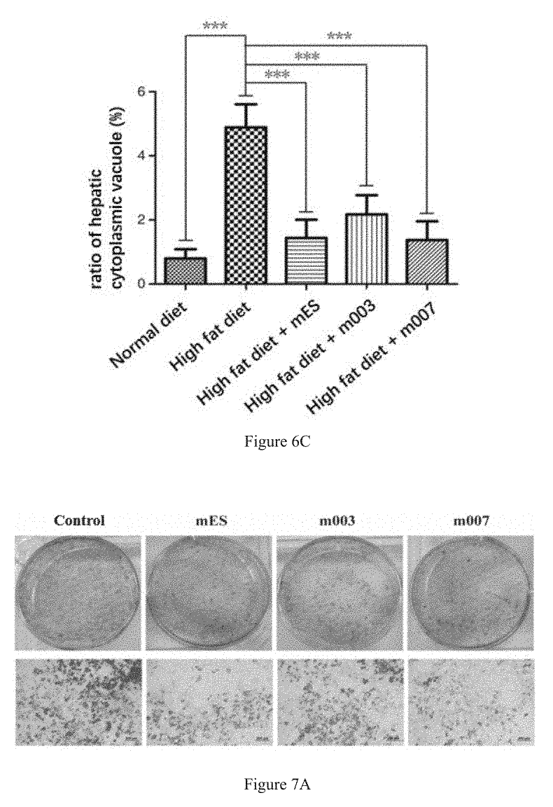

From the mice in Example 5, after the last mice body weight measurement in week 8, the liver tissues were removed and weighed (FIG. 6A, Table 2). The results show that mES, m003, and m007 inhibited the increase in liver weight induced by high-fat diet in mice, with inhibition ratios of 21.3%, 21.3%, and 25.2%, respectively (Table 2).

According to the protocol in example 2, the liver tissues were fixed and embedded in paraffin, stained with HE, observed and recorded conventional optical microscope (Olympus IX71 microscope) (FIG. 6B). HE staining results showed that the fat deposition in livers from mice in mES, m003, and m007 treated groups were significantly lower than that in high-fat diet group without drug administration, with the inhibition ratios of 70.6%, 56.1%, and 73.1%, respectively (FIG. 6C). This indicated that mES, m003, and m007 have a significant inhibitory effect on non-alcoholic fatty liver disease.

### Example 7 PEG-Modified ES and its Variants 003 and 007 (mES, m003, and m007) Significantly Inhibited the Differentiation of Preadipocytes into Adipocytes

3T3-L1 preadipocytes were cultured and induced in the same way as in Example 4. The experiment was divided into 4 groups:

Group 1: control group;

Group 2: mES treated group:

Group 3: m003 treated group:

Group 4: m007 treated group.

Among them, drug treated groups were supplemented with extra 50 μg/ml mES, m003 or m007 during induction (i.e. day 1 to day 8), control group was added with equal volume of protein buffer. Aforesaid drug supplement and control treatment were carried out at each time when the medium was replaced.

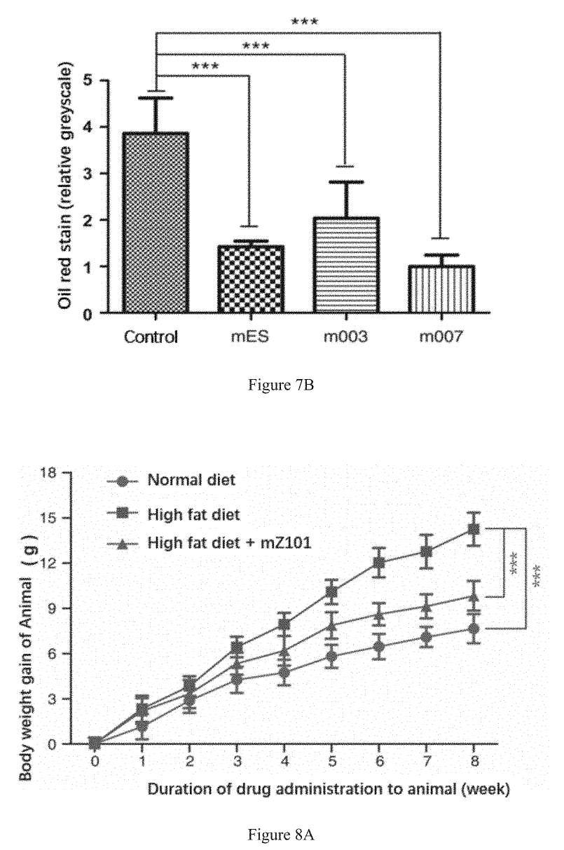

After induction, cells were stained with Oil red according to the experiment method in Example 4. The six-well plate was photographed with a digital camera, also observed and recorded by photograph with an invert microscope (Olympus IX71 microscope) (FIG. 7A). The results showed that mES, m003, and m007 all could directly inhibit the differentiation of preadipocytes into adipocytes, wherein mES and m007 had better inhibition effect than m003 (FIG. 7B). This was consistent with the results of the animal experiment in Example 6, which also explained the reasons why mES and m007 were more effective than m003 in inhibiting weight gain in animals with high-fat diet.

### Example 8 PEG-Modified ES Variant Z101 (mZ101) Significantly Inhibited Weight Gain Induced by High-Fat Diet in Mice

The preparation of experimental mice (8 mice of each group), diet (feedstuff), route of administration, administration cycle and mice body weight measurement were the same as in Example 5. The experiment was grouped as follows:

Group 1: normal diet group:

Group 2: high-fat diet group:

Group 3: high-fat diet+mZ101 treated group (drug treated group).

Wherein the dose was 12 mg/kg/week.

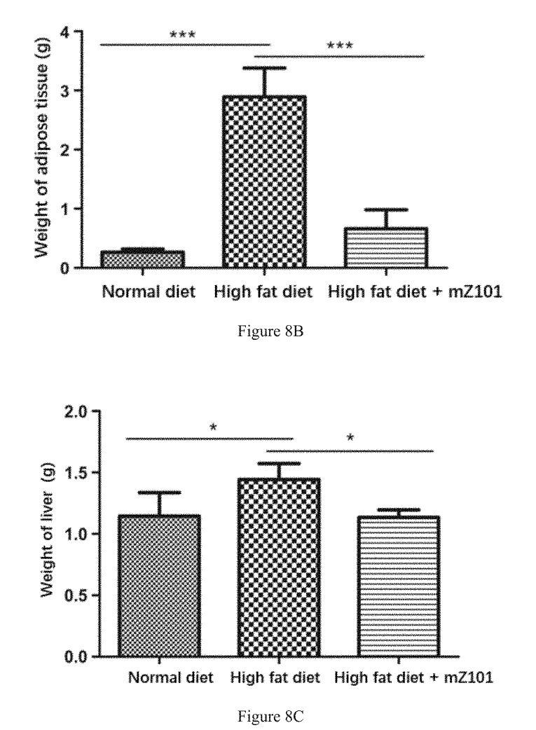

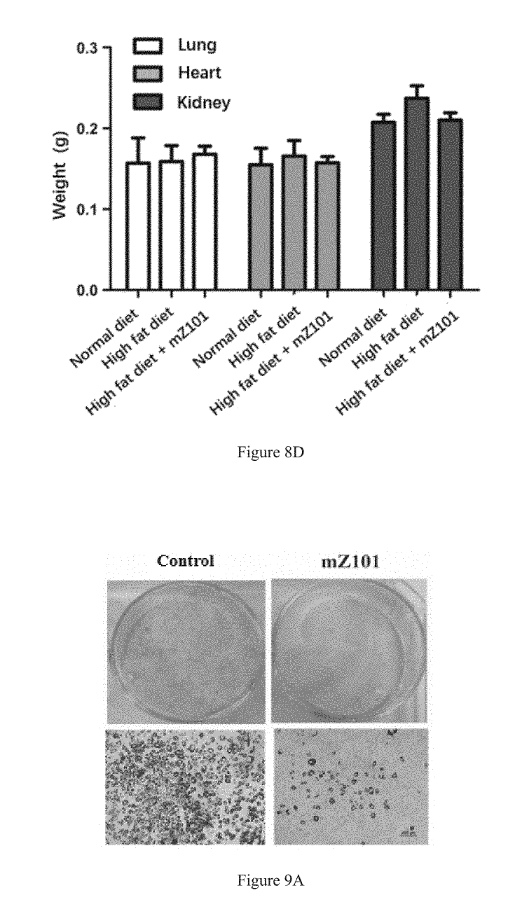

After the last mice body weight measurement in week 8, the body weight curves were plotted (FIG. 8A). The results showed that mZ101 significantly inhibited weight gain due to high-fat diet, and the inhibition ratio was 31% (Table 3).

After the last mice body weight measurement in week 8, the mice were sacrificed and whole body adipose tissues were isolated and weighed (FIGS. 8B and C, Table 3) The results showed that the adipose tissue weight of mice in mZ101 group was significantly lower than that in high-fat diet group without drug treatment, and the inhibition ratio of fat accumulation was 77.2% (Table 3). mZ101 could also inhibit the increase in mice liver weight, and the inhibition ratio was 21.5% (Table 3).

Lungs, heart and kidneys were isolated from mice and weighed (FIG. 8D, Table 3). The results showed that there was no significant difference in weight of mice lungs, heart and kidneys among the three groups, indicating that mZ101 had no effect on lungs, heart and kidneys of mice.

### Example 9 PEG-Modified ES Variant Z101 (mZ101) Significantly Inhibited the Differentiation of Preadipocytes into Adipocytes

3T3-L1 preadipocytes were cultured and induced in the same way as in Example 4. The experiment was divided into 2 groups:

Group 1: control group;

Group 2: mZ101 treated group.

Among them, drug treated group was supplemented with extra 50 μg/ml mZ101 during induction (i.e. day 1 to day 8), control group was added with equal volume of protein buffer. Aforesaid drug supplement and control treatment were carried out at each time when the medium was replaced.

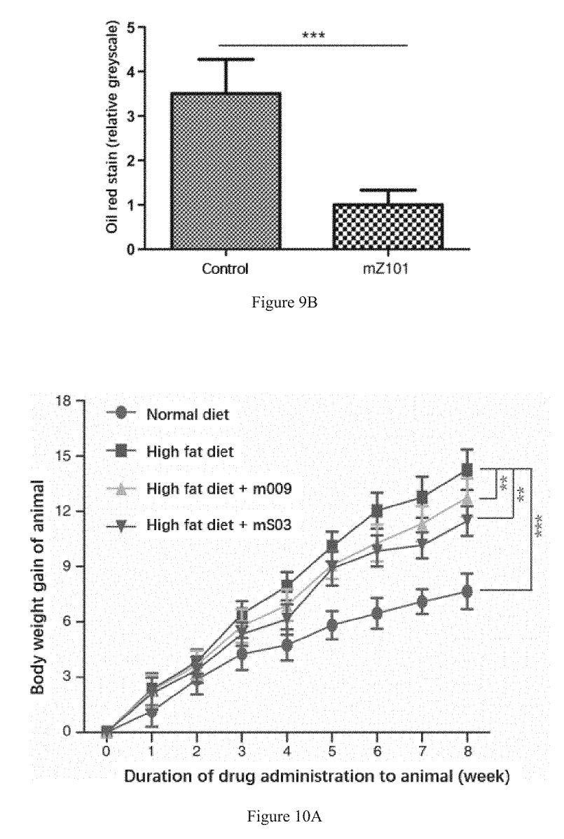

After induction, cells were stained with Oil red according to the experiment method in Example 4. The six-well plate was photographed with a digital camera, also observed and recorded by photograph with an invert microscope (Olympus IX71 microscope) (FIG. 9A). The results showed that mZ101 could directly inhibit the differentiation of preadipocytes into adipocytes (FIG. 9B).

### Example 10 PEG-Modified ES Variants 009 and S03 (m009 and mS03) Significantly Inhibited Weight Gain Induced by High-Fat Diet in Mice, and the Inhibitory Effect of mS03 was Better than that of m009

The preparation of experimental mice (8 mice of each group), diet (feedstuff), route of administration, administration cycle and mice body weight measurement were the same as in Example 5. The experiment was grouped as follows:

Group 1: normal diet group;

Group 2: high-fat diet group;

Group 3: high-fat diet+m009 treated group (drug treated group);

Group 4: high-fat diet+mS03 treated group (drug treated group).

Wherein the dose was 12 mg/kg/week.

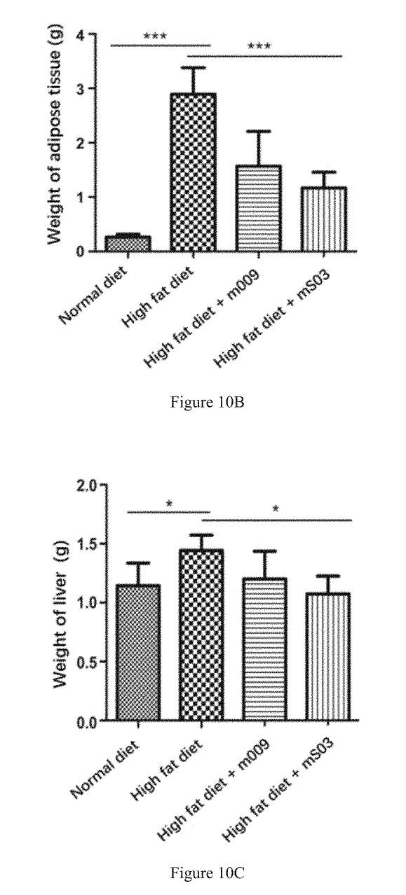

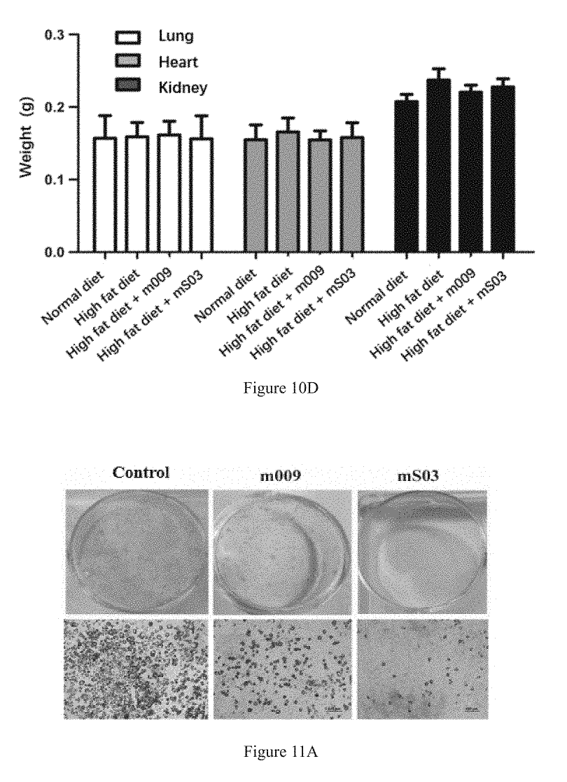

After the last mice body weight measurement in week 8, the body weight curves were plotted (FIG. 10A). The results showed that m009 and mS03 significantly inhibited weight gain due to high-fat diet, and the inhibition ratios were 10.6%, and 19.0% respectively (Table 4).

After the last mice body weight measurement in week 8, the mice were sacrificed and whole body adipose tissues were isolated and weighed (FIGS. 10B and C, Table 4). The results showed that the weight of adipose tissues of mice in m009 and mS03 groups was significantly lower than that in high-fat diet group without drug treatment, and the inhibition ratios of fat accumulation were 45.7%, and 59.5%, respectively (Table 4). m009 and mS03 could also inhibit the increase in mice liver weight, and the inhibition ratios were 16.76%, and 25.7%, respectively (Table 4).

Lungs, heart and kidneys were isolated from mice and weighed (FIG. 10D, Table 4). The results showed that there was no significant difference in weight of mice lungs, heart and kidneys among the four groups, indicating that m009 and mS03 had no effect on lungs, heart and kidneys of mice.

### Example 11 PEG-Modified ES Variants 009 and S03 (m009 and mS03) Significantly Inhibited the Differentiation of Preadipocytes into Adipocytes

3T3-L1 preadipocytes were cultured and induced in the same way as in Example 4. The experiment was divided into 3 groups:

Group 1: control group;

Group 2: m009 treated group:

Group 3: mS03 treated group.

Among them, drug treated groups were supplemented with extra 50 μg/ml m009 or mS03 during induction (i.e. day 1 to day 8), control group was added with equal volume of protein buffer. Aforesaid drug supplement and control treatment were carried out at each time when the medium was replaced.

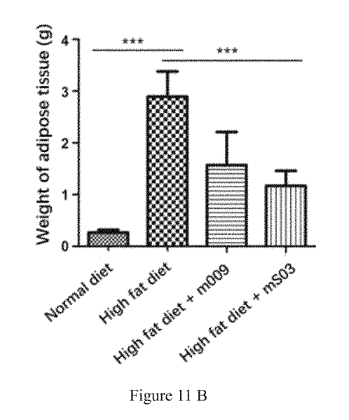

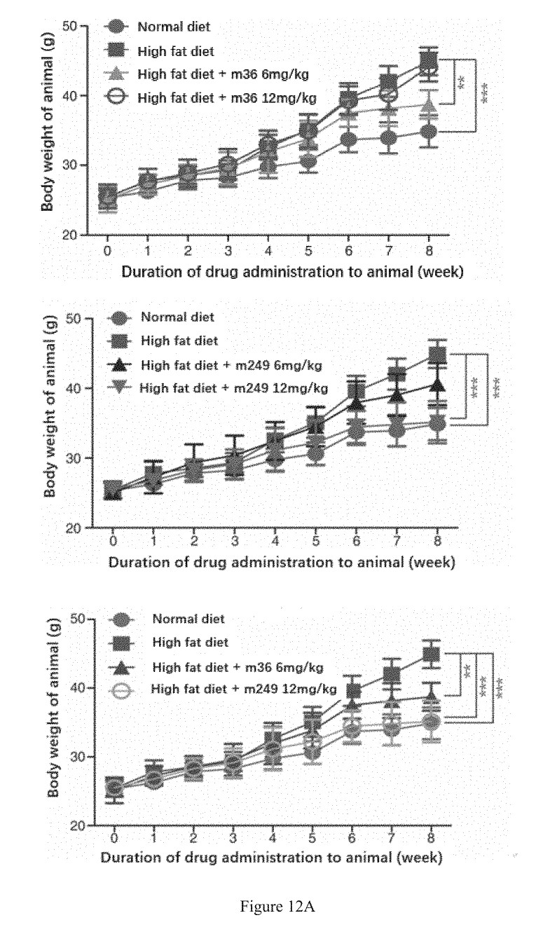

After induction, cells were stained with Oil red according to the experiment method in Example 4. The six-well plate was photographed with a digital camera, also observed and recorded by photograph with an invert microscope (Olympus IX71 microscope) (FIG. 11A). The results showed that both m009 and mS03 could directly inhibit the differentiation of preadipocytes into adipocytes, wherein the mS03 had better inhibition effect than m009 (FIG. 11B). This was consistent with the results of the animal experiment in Example 9, which also explained the reasons why mS03 was more effective than m009 in inhibiting weight gain in animals with high-fat diet.

### Example 12 PEG-Modified ES Variants 36 and 249 (m36 and m249) Significantly Inhibited Weight Gain Induced by High-Fat Diet in Mice

The preparation of experimental mice (8 mice of each group), diet (feed), route of administration, administration cycle and mice body weight measurement were the same as in Example 5. The experiment was grouped as follows:

Group 1: normal diet group;

Group 2: high-fat diet group;

Group 3: high-fat diet+m36 (6 mg/kg/week) treated group (drug treated group);

Group 4: high-fat diet+m36 (12 mg/kg/week) treated group (drug treated group):

Group 5: high-fat diet+m249 (6 mg/kg/week) treated group (drug treated group);

Group 6: high-fat diet+m249 (12 mg/kg/week) treated group (drug treated group).

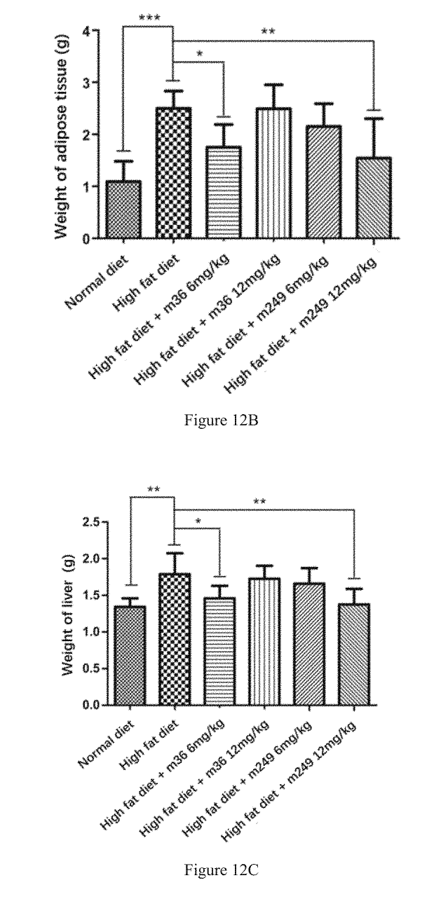

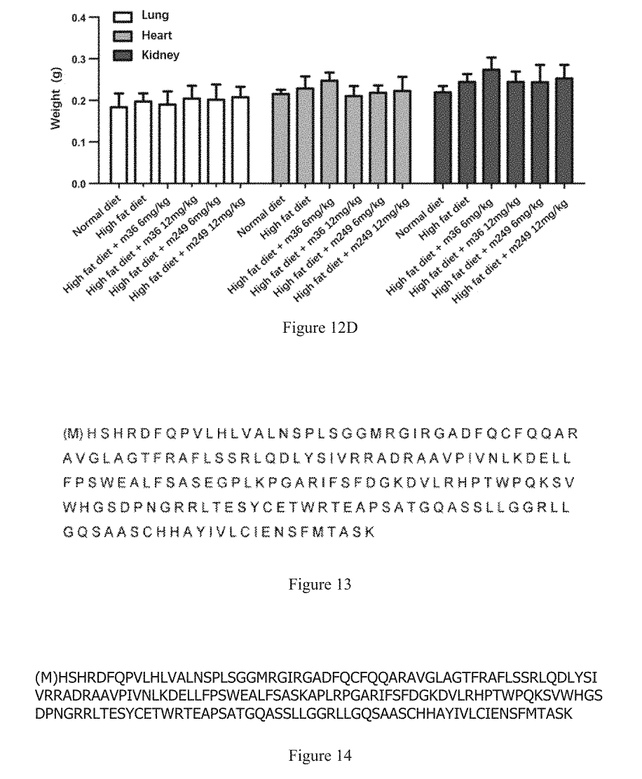

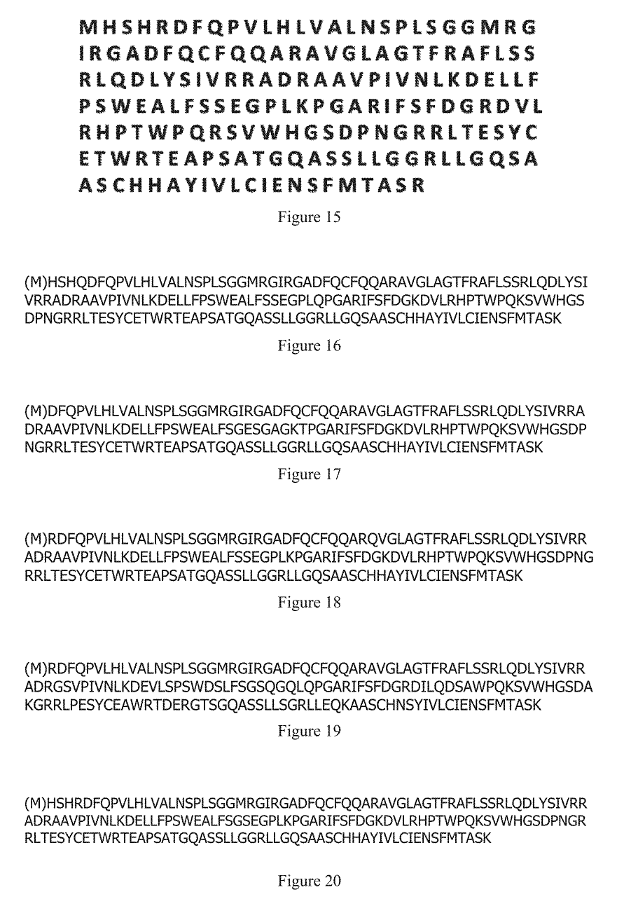

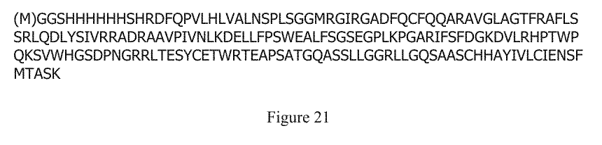

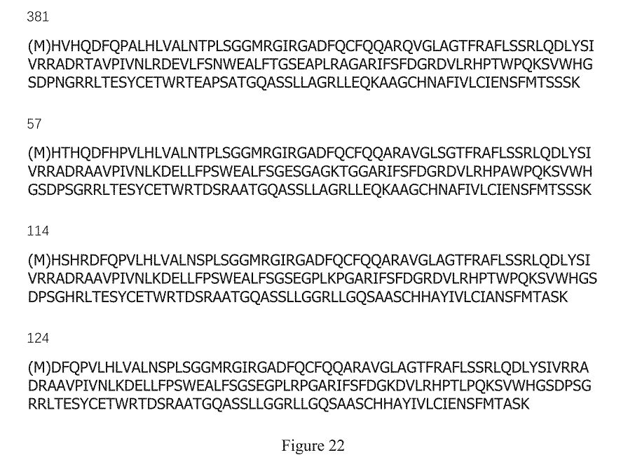

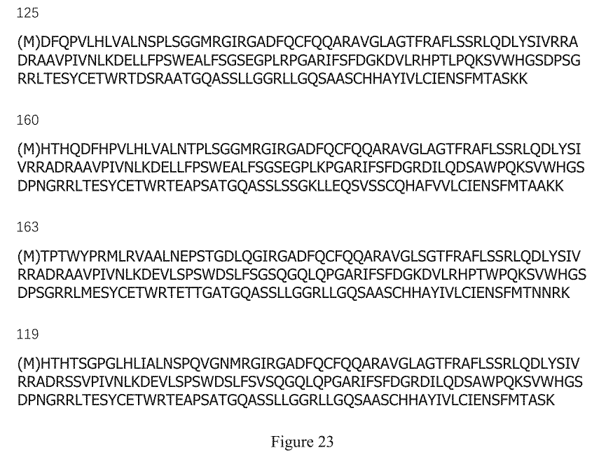

After the last mice body weight measurement in week 8, the body weight curves were plotted (FIG. 12A). The results showed that low-dose m36 (6 mg/kg/week) and high-dose m249 (12 mg/kg/week) significantly inhibited weight gain due to high-fat diet, and the inhibition ratios were 30.3%, and 50.3%, respectively (Table 5).

After the last mice body weight measurement in week 8, the mice were sacrificed and whole body adipose tissues were isolated and weighed (FIGS. 12B and C, Table 5). The results showed that the weight of adipose tissues of mice in low-dose m36 (6 mg/kg/week) and high-dose m249 (12 mg/kg/week) groups was significantly lower than that in high-fat diet group without drug treatment, and the inhibition ratios of low-dose m36 (6 mg/kg/week) and high-dose m249 (12 mg/kg/week) on fat accumulation induced by high-fat diet in mice were 30%, and 38.4%, respectively (Table 5). Low-dose m36 (6 mg/kg/week) and high-dose m249 (12 mg/kg/week) could also inhibit the increase in mice liver weight, and the inhibition ratios were 18.4%, and 22.9%, respectively (Table 5).

Lungs, heart and kidneys were isolated from mice and weighed (FIG. 12D). The results showed that there was no significant difference in the weight of lungs, heart and kidneys among the six groups, indicating that m36 and m249 had no effect on lungs, heart and kidneys of mice.

TABLE 1

| | | | Group of | Group of |
|---|---|---|---|---|
| | Group of | Group of | high-fat diet + | high-fat diet + |
| | normal diet | high-fat diet | ES | YH-16 |
| Mice body weight before | 22.4 ± 0.83 | 23.5 ± 0.88 | 23.4 ± 1.25 | 23.5 ± 1.04 |
| administration (g) | | | | |
| Mice body weight after | 28.1 ± 0.89 | 37.9 ± 1.35 | 32.4 ± 0.83 | 33.5 ± 0.97 |
| administration (g) | | | | |
| Mice body weight gain (g) | 5.7 ± 0.79 | 14.4 ± 2.51 | 9.0 ± 1.84 | 10.00 ± 1.61 |
| Mice adipose tissue weight | 0.66 ± 0.13 | 2.89 ± 0.59 | 1.51 ± 0.67 | 1.67 ± 0.48 |
| after administration (g) | | | | |
| Mice liver weight after | 0.86 ± 0.15 | 1.22 ± 0.18 | 0.93 ± 0.14 | 0.97 ± 0.12 |
| administration (g) | | | | |
| Mice heart weight after | 0.133 ± 0.006 | 0.131 ± 0.007 | 0.132 ± 0.008 | 0.138 ± 0.009 |
| admistration (g) | | | | |
| Mice lung weight after | 0.141 ± 0.018 | 0.142 ± 0.008 | 0.142 ± 0.006 | 0.147 ± 0.012 |
| administration (g) | | | | |
| Mice kidney weight after | 0.181 ± 0.014 | 0.18 ± 0.01 | 0.189 ± 0.015 | 0.191 ± 0.010 |
| administration (g) | | | | |

TABLE 2

| | | | Group of | Group of | Group of |
|---|---|---|---|---|---|
| | Group of | Group of | high-fat diet + | high-fat diet + | high-fat diet + |
| | normal diet | high-fat diet | mES | m003 | m007 |
| Mice body weight | 22.4 ± 0.83 | 24.5 ± 1.06 | 24.5 ± 1.14 | 23.6 ± 1.09 | 22.9 ± 1.38 |
| before administration | | | | | |
| (g) | | | | | |
| Mice body weight after | 27.5 ± 0.94 | 38.5 ± 1.06 | 33.8 ± 0.72 | 34.4 ± 1.38 | 31.2 ± 1.25 |
| administration (g) | | | | | |
| Mice body weight gain | 5.1 ± 0.87 | 14.0 ± 0.82 | 9.28 ± 0.91 | 10.8 ± 1.95 | 7.99 ± 0.95 |
| (g) | | | | | |
| Mice adipose tissue | 0.66 ± 0.13 | 3.04 ± 0.30 | 1.78 ± 0.43 | 1.81 ± 0.59 | 2.07 ± 0.81 |
| weight after | | | | | |
| administration (g) | | | | | |
| Mice liver weight after | 0.86 ± 0.15 | 1.27 ± 0.16 | 1.00 ± 0.10 | 0.95 ± 0.18 | 1.00 ± 0.18 |
| administration (g) | | | | | |
| Mice heart weight after | 0.14 ± 0.020 | 0.15 ± 0.008 | 0.15 ± 0.012 | 0.15 ± 0.017 | 0.14 ± 0.010 |
| administration (g) | | | | | |
| Mice lung weight after | 0.133 ± 0.006 | 0.134 ± 0.006 | 0.134 ± 0.005 | 0.132 ± 0.012 | 0.130 ± 0.007 |
| administration (g) | | | | | |
| Mice kidney weight | 0.181 ± 0.014 | 0.185 ± 0.014 | 0.181 ± 0.018 | 0.186 ± 0.014 | 0.186 ± 0.014 |
| after administration (g) | | | | | |

TABLE 3

| | | | Group of |
|---|---|---|---|
| | Group of | Group of | high-fat diet + |
| | normal diet | high-fat diet | mZ101 |
| Mice body weight before | 20.9 ± 0.40 | 21.1 ± 0.42 | 20.7 ± 0.31 |
| administration (g) | | | |
| Mice body weight after | 28.5 ± 0.75 | 35.3 ± 1.75 | 30.6 ± 2.21 |
| administration (g) | | | |
| Mice body weight gain (g) | 7.6 ± 0.97 | 14.2 ± 1.09 | 9.8 ± 0.98 |
| Mice adipose tissue weight | 0.26 ± 0.05 | 2.89 ± 0.49 | 0.66 ± 0.32 |
| after administration (g) | | | |
| Mice liver weight after | 1.14 ± 0.19 | 1.44 ± 0.13 | 1.13 ± 0.06 |
| administration (g) | | | |
| Mice heart weight after | 0.145 ± 0.018 | 0.163 ± 0.014 | 0.155 ± 0.007 |
| administration (g) | | | |
| Mice lung weight after | 0.16 ± 0.031 | 0.16 ± 0.019 | 0.17 ± 0.010 |
| administration (g) | | | |
| Mice kidney weight after | 0.21 ± 0.010 | 0.24 ± 0.015 | 0.21 ± 0.009 |
| administration (g) | | | |

TABLE 4
(FIG. 11)

| | | | Group of | Group of |
|---|---|---|---|---|
| | Group of | Group of | high-fat diet + | high-fat diet + |
| | normal diet | high-fat diet | m009 | mS03 |
| Mice body weight before | 20.9 ± 0.40 | 21.1 ± 0.42 | 20.8 ± 0.35 | 20.9 ± 0.44 |
| administration (g) | | | | |
| Mice body weight after | 28.5 ± 0.75 | 35.3 ± 1.75 | 33.5 ± 1.80 | 32.4 ± 2.35 |
| administration (g) | | | | |
| Mice body weight gain (g) | 7.6 ± 0.97 | 14.2 ± 1.09 | 12.7 ± 1.03 | 11.5 ± 0.80 |
| Mice adipose tissue weight | 0.26 ± 0.05 | 2.89 ± 0.49 | 1.57 ± 0.64 | 1.17 ± 0.29 |
| after administration (g) | | | | |
| Mice liver weight after | 1.14 ± 0.19 | 1.44 ± 0.13 | 1.20 ± 0.24 | 1.07 ± 0.15 |
| administration (g) | | | | |
| Mice heart weight after | 0.145 ± 0.018 | 0.163 ± 0.014 | 0.156 ± 0.011 | 0.159 ± 0.020 |
| administration (g) | | | | |
| Mice lung weight after | 0.16 ± 0.031 | 0.16 ± 0.019 | 0.16 ± 0.019 | 0.16 ± 0.032 |
| administration (a) | | | | |
| Mice kidney weight after | 0.21 ± 0.010 | 0.24 ± 0.015 | 0.22 ± 0.010 | 0.23 ± 0.011 |
| administration (g) | | | | |

TABLE 5
(FIG. 12)

| | | | Group of | Group of | Group of | Group of |
|---|---|---|---|---|---|---|
| | | | high-fat diet + | high-fat diet + | high-fat diet + | high-fat diet + |
| | Group of | Group of | 6 mg/kg/week | 12 mg/kg/week | 6 mg/kg/week | 12 mg/kg/week |
| | normal diet | high-fat diet | of m36 | of m36 | of m249 | of m249 |
| Mice body weight before | 25.4 ± 1.06 | 25.4 ± 1.24 | 25.1 ± 1.87 | 25.5 ± 1.71 | 25.0 ± 0.64 | 25.5 ± 0.98 |
| administration (g) | | | | | | |
| Mice body weight after | 34.9 ± 2.31 | 44.9 ± 2.02 | 38.7 ± 2.02 | 44.1 ± 2.04 | 40.6 ± 3.03 | 35.2 ± 3.03 |
| administration (g) | | | | | | |
| Mice body weight gain (g) | 9.5 ± 2.64 | 19.5 ± 2.43 | 13.6 ± 3.73 | 18.6 ± 3.06 | 15.6 ± 2.48 | 9.7 ± 2.42 |
| Mice adipose tissue weight | 1.09 ± 0.39 | 2.50 ± 0.33 | 1.75 ± 0.44 | 2.49 ± 0.46 | 2.15 ± 0.44 | 1.54 ± 0.76 |
| after administration (g) | | | | | | |
| Mice liver weight after | 1.34 ± 0.12 | 1.79 ± 0.29 | 1.46 ± 0.17 | 1.73 ± 0.18 | 1.66 ± 0.21 | 1.38 ± 0.21 |
| administration (g) | | | | | | |
| Mice heart weight after | 0.21 ± 0.010 | 0.23 ± 0.029 | 0.25 ± 0.020 | 0.21 ± 0.024 | 0.22 ± 0.018 | 0.22 ± 0.034 |
| administration (g) | | | | | | |
| Mice lung weight after | 0.183 ± 0.033 | 0.197 ± 0.020 | 0.190 ± 0.032 | 0.204 ± 0.031 | 0.201 ± 0.037 | 0.208 ± 0.025 |
| administration (g) | | | | | | |
| Mice kidney weight after | 0.22 ± 0.015 | 0.24 ± 0.019 | 0.27 ± 0.029 | 0.24 ± 0.025 | 0.24 ± 0.042 | 0.25 ± 0.033 |
| administration (g) | | | | | | |

The names and the corresponding amino acid sequences of ES and its mutants according to the present invention are as follows:

ES
(SEQ ID NO: 1)
(M)HSHRDFQPVLHLVALNSPLSGGMRGIRGADFQCFQQARAVGLAGTFR
AFLSSRLQDLYSIVRRADRAAVPIVNLKDELLFPSWEALFSGSEGPLKPG
ARIFSFDGKDVLRHPTWPQKSVWHGSDPNGRRLTESYCETWRTEAPSATG
QASSLLGGRLLGQSAASCHHAYIVLCIENSFMTASK
YH16
(SEQ ID NO: 2)
(M)GGSHHHHHHSHRDFQPVLHLVALNSPLSGGMRGIRGADFQCFQQARA
VGLAGTFRAFLSSRLQDLYSIVRRADRAAVPIVNLKDELLFPSWEALFSG
SEGPLKPGARIFSFDGKDVLRHPTWPQKSVWHGSDPNGRRLTESYCETWR
TEAPSATGQASSLLGGRLLGQSAASCHHAYIVLCIENSFMTASK
003
(SEQ ID NO: 3)
(M)HSHRDFQPVLHLVALNSPLSGGMRGIRGADFQCFQQARAVGLAGTFR
AFLSSRLQDLYSIVRRADRAAVPIVNLKDELLFPSWEALFSASEGPLKPG
ARIFSFDGKDVLRHPTWPQKSVWHGSDPNGRRLTESYCETWRTEAPSATG
QASSLLGGRLLGQSAASCHHAYIVLCIENSFMTASK
007
(SEQ ID NO: 4)
(M)HSHRDFQPVLHLVALNSPLSGGMRGIRGADFQCFQQARAVGLAGTFR
AFLSSRLQDLYSIVRRADRAAVPIVNLKDELLFPSWEALFSASKAPLQPG
ARIFSFDGKDVLRHPTWPQKSVWHGSDPNGRRLTESYCETWRTEAPSATG
QASSLLGGRLLGQSAASCHHAYIVLCIENSFMTASK
Z101
(SEQ ID NO: 5)
(M)HSHRDFQPVLHLVALNSPLSGGMRGIRGADFQCFQQARAVGLAGTFR
AFLSSRLQDLYSIVRRADRAAVPIVNLKDELLFPSWEALFSSEGPLKPGA
RIFSFDGRDVLRHPTWPQRSVWHGSDPNGRRLTESYCETWRTEAPSATGQ
ASSLLGGRLLGQSAASCHHAYIVLCIENSFMTASR
009
(SEQ ID NO: 6)
(M)HSHQDFQPVLHLVALNSPLSGGMRGIRGADFQCFQQARAVGLAGTFR
AFLSSRLQDLYSIVRRADRAAVPIVNLKDELLFPSWEALFSSEGPLQPGA
RIFSFDGKDVLRHPTWPQKSVWHGSDPNGRRLTESYCETWRTEAPSATGQ
ASSLLGGRLLGQSAASCHHAYIVLCIENSFMTASK
S03
(SEQ ID NO: 7)
(M)DFQPVLHLVALNSPLSGGMRGIRGADFQCFQQARAVGLAGTFRAFLS
SRLQDLYSIVRRADRAAVPIVNLKDELLFPSWEALFSGESGAGKTPGARI
FSFDGKDVLRHPTWPQKSVWHGSDPNGRRLTESYCETWRTEAPSATGQAS
SLLGGRLLGQSAASCHHAYIVLCIENSFMTASK
36
(SEQ ID NO: 8)
(M)RDFQPVLHLVALNSPLSGGMRGIRGADFQCFQQARQVGLAGTFRAFL
SSRLQDLYSIVRRADRAAVPIVNLKDELLFPSWEALFSSEGPLKPGARIF
SFDGKDVLRHPTWPQKSVWHGSDPNGRRLTESYCETWRTEAPSATGQASS
LLGGRLLGOSAASCHHAYIVLCIENSFMTASK
249
(SEQ ID NO: 9)
(M)RDFQPVLHLVALNSPLSGGMRGIRGADFQCFQQARAVGLAGTFRAFL
SSRLQDLYSIVRRADRGSVPIVNLKDEVLSPSWDSLFSGSQGQLQPGARI
FSFDGRDILQDSAWPQKSVWHGSDAKGRRLPESYCEAWRTDERGTSGQAS
SLISGRLLEQKAASCHNSYIVLCIENSFMTASK
381
(SEQ ID NO: 10)
(M)HVHQDFQPALHLVALNTPLSGGMRGIRGADFQCFQQARQVGLAGTFR
AFLSSRLQDLYSIVRRADRTAVPIVNLRDEVLFSNWEALFTGSEAPLRAG
ARIFSFDGRDVLRHPTWPQKSVWHGSDPNGRRLTESYCETWRTEAPSATG
QASSLLAGRLLEQKAAGCHNAFIVLCIENSFMTSSSK
57
(SEQ ID NO: 11)
(M)HTHQDFHPVLHLVALNTPLSGGMRGIRGADFQCFQQARAVGLSGTFR
AFLSSRLQDLYSIVRRADRAAVPIVNLKDELLFPSWEALFSGESGAGKTG
GARIFSFDGRDVLRHPAWPQKSVWHGSDPSGRRLTESYCETWRTDSRAAT
GQASSLLAGRLLEQKAAGCHNAFIVLCIENSFMTSSSK
114
(SEQ ID NO: 12)
(M)HSHRDFQPVLHLVALNSPLSGGMRGIRGADFQCFQQARAVGLAGTFR
AFLSSRLQDLYSIVRRADRAAVPIVNLKDELLFPSWEALFSGSEGPLKPG
ARIFSFDGRDVLRHPTWPQKSVWHGSDPSGHRLTESYCETWRTDSRAATG
QASSLLGGRLLGQSAASCHHAYIVLCIANSFMTASK
124
(SEQ ID NO: 13)
(M)DFQPVLHLVALNSPLSGGMRGIRGADFQCFQQARAVGLAGTFRAFLS
SRLQDLYSIVRRADRAAVPIVNLKDELLFPSWEALFSGSEGPLRPGARIF
SFDGKDVLRHPTLPQKSVWHGSDPSGRRLTESYCETWRTDSRAATGQASS
LLGGRLLGQSAASCHHAYIVLCIENSFMTASK
125
(SEQ ID NO: 14)
(M)DFQPVLHLVALNSPLSGGMRGIRGADFQCFQQARAVGLAGTFRAFLS
SRLQDLYSIVRRADRAAVPIVNLKDELLFPSWEALFSGSEGPLRPGARIF
SFDGKDVLRHPTLPQKSVWHGSDPSGRRLTESYCETWRTDSRAATGQASS
LLGGRLLGQSAASCHHAYIVLCIENSFMTASKK
160
(SEQ ID NO: 15)
(M)HTHQDFHPVLHLVALNTPLSGGMRGIRGADFQCFQQARAVGLAGTFR
AFLSSRLQDLYSIVRRADRAAVPIVNLKDELLFPSWEALFSGSEGPLKPG
ARIFSFDGRDILQDSAWPQKSVWHGSDPNGRRLTESYCETWRTEAPSATG
QASSLSSGKLLEQSVSSCQHAFVVLCIENSFMTAAKK
119
(SEQ ID NO: 16)
(M)HTHTSGPGLHLIALNSPQVGNMRGIRGADFQCFQQARAVGLAGTFRA
FLSSRLQDLYSIVRRADRSSVPIVNLKDEVLSPSWDSLFSVSQGQLQPGA
RIFSFDGRDILQDSAWPQKSVWHGSDPNGRRLTESYCETWRTEAPSATGQ
ASSLLGGRLLGQSAASCHHAYIVLCIENSFMTASK
163
(SEQ ID NO: 17)
(M)TPTWYPRMLRVAALNEPSTGDLOGIRGADFQCFQQARAVGLSGTFRA
FLSSRLQDLYSIVRRADRAAVPIVNLKDEVLSPSWDSLFSGSQGQLQPGA
RIFSFDGKDVLRHPTWPQKSVWHGSDPSGRRLMESYCETWRTETTGATGQ
ASSLLGGRLLGQSAASCHHAYIVLCIENSFMTNNRK

1-6. (canceled)

7. A method for treating dietary obesity, non-alcoholic fatty liver disease, insulin resistance or glucose intolerance, comprising administering to a subject a therapeutically effective amount of endostatin or a functional variant thereof.

8. The method of claim 7, wherein the functional variant is selected from the group consisting of: YH-16, 003, 007, Z101, ES006, ES008, ES011, S02, S09, Z006, Z008, ZN1, 009, S03, 36, 249, 381, 57, 114, 124, 125, 160, 163, 119, mES, mYH-16, m003, m007, mZ101, mES006, mES008, mES011, mS02, mS09, mZ006, mZ008, mZN1, m009, mS03, m36, m249, m381, m57, m114, m124, m125, m160, m163, and m119.

9. The method of claim 7, wherein the functional variant is selected from the group consisting of: YH-16, 003, 007, Z101, 009, S03, 36, 249, mES, mYH-16, m003, m007, mZ101, m009, mS03, m36, and m249.

10. A method for inhibiting adipocyte differentiation, comprising administering to a subject a therapeutically effective amount of endostatin or a functional variant thereof.

11. The method of claim 10, wherein the functional variant is selected from the group consisting of: YH-16, 003, 007, Z101, ES006, ES008, ES011, S02, S09, Z006, Z008, ZN1, 009, S03, 36, 249, 381, 57, 114, 124, 125, 160, 163, 119, mES, mYH-16, m003, m007, mZ101, mES006, mES008, mES011, mS02, mS09, mZ006, mZ008, mZN1, m009, mS03, m36, m249, m381, m57, m114, m124, m125, m160, m163, and m119.

12. The method of claim 10, wherein the functional variant is selected from the group consisting of: YH-16, 003, 007, Z101, 009, S03, 36, 249, mES, mYH-16, m003, m007, mZ101, m009, mS03, m36, and m249.

13. A medicament for treating dietary obesity, non-alcoholic fatty liver disease, insulin resistance or glucose intolerance, comprising endostatin or a functional variant thereof as active ingredient.

14. The medicament of claim 13, wherein the functional variant is selected from the group consisting of: YH-16, 003, 007, Z101, ES006, ES008, ES011, S02, S09, Z006, Z008, ZN1, 009, S03, 36, 249, 381, 57, 114, 124, 125, 160, 163, 119, mES, mYH-16, m003, m007, mZ101, mES006, mES008, mES011, mS02, mS09, mZ006, mZ008, mZN1, m009, mS03, m36, m249, m381, m57, m114, m124, m125, m160, m163, and m119.

15. The medicament of claim 13, wherein the functional variant is selected from the group consisting of: YH-16, 003, 007, Z101, 009, S03, 36, 249, mES, mYH-16, m003, m007, mZ101, m009, mS03, m36, and m249.
